(19) United States
(12) Patent Application Publication
(10) Pub. No.: US 2008/0312212 A1
(43) Pub. Date: Dec. 18, 2008

(54) Organic Compounds

(75) Inventors: Stephen Paul Collingwood, West Sussex (GB); Nichola Smith, West Sussex (GB)

Correspondence Address: NOVARTIS INSTITUTES FOR BIOMEDICAL RESEARCH, INC., 400 TECHNOLOGY SQUARE, CAMBRIDGE, MA, 02139, US

(73) Assignee: Novartis AG

(21) Appl. No.: 12/158,803

(22) PCT Filed: Dec. 20, 2006
(86) PCT No.: PCT/EP06/12320

§ 371 Date: Jun. 23, 2008

(30) Foreign Application Priority Data
Dec. 22, 2005 (GB) 0526244.9

(51) Int. Cl. A61K 31/55; C07D 403/02; C07D 401/02; A61K 31/4965; A61P 11/06; A61P 37/00; A61P 37/08; C07D 241/20; A61K 31/497; C07D 223/22

(52) U.S. Cl. 514/217; 544/405; 544/407; 540/589; 514/255.05; 514/255.06

(57) ABSTRACT

A compound of formula (I)

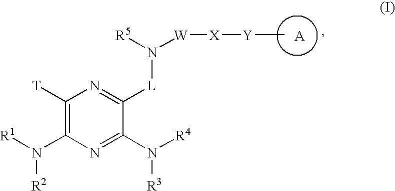

or tautomers, or stereoisomers, or solvates, or pharmaceutically acceptable salts thereof, wherein R1, R2, R3, R4, R5, T, L, W, X, Y and A are as defined herein for the for treatment of conditions mediated by the blockade of an epithelial sodium channel, particularly an inflammatory or allergic condition.

This invention relates to organic compounds, their preparation and use as pharmaceuticals.

In one aspect, the present invention provides compounds of formula (I)

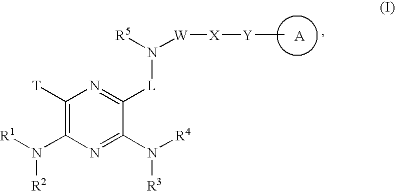

or tautomers, or stereoisomers, or solvates, or pharmaceutically acceptable salts thereof, wherein
R¹, R², R³, and R⁴ are independently selected from H, C₁-C₈-alkyl, C₁-C₈-alkyl-carboxy, C₁-C₈-haloalkyl, C₃-C₁₅-carbocyclic group, C₁-C₈-alkylcarbonyl, C₁-C₈-alkoxycarbonyl, , a C₆-C₁₅-membered aromatic carbocyclic group, a 3- to 14-membered heterocyclic group, a C₁-C₈-alkyl substituted by a 3- to 14-membered heterocyclic group, and a C₁-C₈-alkyl substituted by a C₆-C₁₅-membered aromatic carbocyclic group,
   or R¹ and R² with the nitrogen atom to which they are attached form a C₃-C₁₄-membered heterocyclic group optionally substituted by R¹⁴,
   or R³ and R⁴ with the nitrogen atom to which they are attached form a C₃-C₁₄-membered heterocyclic group optionally substituted by R¹⁴;
L is selected from

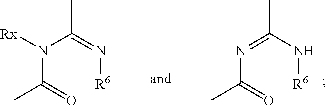

R⁶, R⁵ and R^{x} are selected from H and C₁-C₈ alkyl, C₁-C₈-alkyl-carboxy, C₁-C₈-alkyl-alkoxy, C₁-C₈-haloalkyl, C₃-C₁₅-carbocyclic group, C₁-C₈-alkylcarbonyl, C₁-C₈-alkoxycarbonyl, nitro, cyano, a C₆-C₁₅-membered aromatic carbocyclic group, a 3- to 14-membered heterocyclic group, a C₁-C₈-alkyl substituted by a 3- to 14-membered heterocyclic group, and a C₁-C₈-alkyl substituted by a C₆-C₁₅-membered aromatic carbocyclic group;
W is selected from C₁-C₇ alkylene;
X is selected from —NR⁷(C═O)—,
   —NR⁷(C═O)NR⁷—,
   —NR⁸SO₂—,
   —NR⁸(SO₂)NR⁸—,
   —NR⁷(C═O)O—,
   —O(C═O)—,
   —O(C═O)O—,
   —O(C═O)NR⁷—,
   —(C═O)NR⁷—,
   —(C═O)O—,
   —(SO₂)NR⁸—, and
   —(SO₂)NR⁸-Z-(SO₂)NR⁸;
Y is —C₀-C₈ alkylene- or —(C₀-C₈-alkylene)-SO₂NH—;
Z is C₁-C₄alkylene;
where W, Y and Z are optionally substituted by C₁-C₈-alkyl, halogen, C₁-C₈-alkoxy, carboxy, C₁-C₈-alkyl-carboxy, C₁-C₈-haloalkyl, C₁-C₈-haloalkoxy, C₃-C₁₅-carbocyclic group, C₁-C₈-alkylcarbonyl, C₁-C₈-alkoxycarbonyl, nitro, cyano, a C₃-C₁₅-carbocyclic group, a C₆-C₁₅-membered aromatic carbocyclic group, a C₁-C₈-alkyl substituted by a C₆-C₁₅-membered aromatic carbocyclic group, a 3- to 14-membered heterocyclic group containing at least one ring heteroatom selected from the group consisting of nitrogen, oxygen and sulphur, and a C₁-C₈-alkyl substituted by a 4- to 14-membered heterocyclic group containing at least one ring heteroatom selected from the group consisting of nitrogen, oxygen and sulphur;

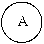

is a C₆-C₁₅-membered aromatic carbocyclic group and a 4- to 14-membered heterocyclic group;
R⁷, R⁸, R¹¹ and R¹², are independently selected from H, C₁-C₈-alkyl, C₁-C₈-alkyl substituted by a C₆-C₁₅-membered aromatic carbocyclic group, C₁-C₈-haloalkyl and a 5- to 14-membered heterocyclic group; R⁷ and R⁸, independently, by way of a C₁ to C₄ alkyl group can form a bond with a carbon atom of group W or Y to create a 5- to 14-membered heterocyclic group;
T is selected from H, halogen, C₁-C₈ alkyl, C₁-C₈-haloalkyl, C₁-C₈-haloalkoxy, C₃-C₁₅-carbocyclic group, nitro, cyano, a C₆-C₁₅-membered aromatic carbocyclic group, a and a C₁-C₈-alkyl substituted by a C₆-C₁₅-membered aromatic carbocyclic group;
wherein each C₆-C₁₅-membered aromatic carbocyclic group and each 4 to 14 membered heterocyclic group, unless otherwise specified is independently optionally substituted by one or more groups selected from OH, C₁-C₈-alkoxy, C₁-C₈-alkyl, halogen, SO₂NR¹¹R¹², hydroxyC₁-C₈-alkoxy, optionally substituted by hydroxyl, (C₀₋₄alkylene) CONR¹¹R¹², (C₀₋₄alkylene) N═C(NR¹¹R¹²)₂, —O—(C₁₋₄alkylene)-N═C(NR¹¹R¹²)₂, —O—(C₁₋₄alkylene)-CONR¹¹R¹², C₆-C₁₀-aralkoxy, C₇-C₁₀-aralkyl, SH, S(C₁₋₈alkylene), SO₂ (C₁₋₈alkylene) SO(C₁₋₁₈alkylene), NR¹¹R¹², R¹⁵, a C₁-C₈-alkyl substituted by R¹⁵, R¹⁶, a C₁-C₈-alkyl substituted by R¹⁶, O(C₁-C₈-alkylene)-NR¹¹C(C═O)O—(C₀-C₄-alkylene)-R¹⁵, cyano, oxo, carboxy, nitro, C₁-C₈-alkylcarbonyl, hydroxy-C₁-C₈-alkyl, C₁-C₈-haloalkyl, amino-C₁-C₈-alkyl, amino(hydroxy)C₁-C₈-alkyl and C₁-C₈-alkoxy optionally substituted by aminocarbonyl;
and wherein each alkylene group, unless otherwise specified, is optionally substituted by C₁-C₈-alkyl, halogen, C₁-C₈-alkoxy, carboxy, C₁-C₈-alkyl-carboxy, C₁-C₈-haloalkyl, C₁-C₈-haloalkoxy, C₃-C₁₅-carbocyclic group, C₁-C₈-alkylcarbonyl, C₁-C₈-alkoxycarbonyl, nitro, cyano, R¹⁵, a C₁-C₈-alkyl substituted by R¹⁵, R¹⁶ or a C₁-C₈-alkyl substituted by R¹⁶;
R¹⁴ is selected from H, halogen, C₁-C₈-alkyl, OH, C₆-C₁₅-membered aromatic carbocyclic group, C₇-C₁₄-aralkyl, and O—C₇-C₁₄-aralkyl;
R¹⁵ is a C₆-C₁₅-membered aromatic carbocyclic group, optionally substituted by OH, C₁-C₈-alkoxy, C₁-C₈-alkyl, halogen and C₁-C₈-haloalkyl; and
R¹⁶ is a 3 to 14 membered heterocyclic group, optionally substituted by OH, C₁-C₈-alkoxy, C₁-C₈-alkyl, halogen and C₁-C₈-haloalkyl.

In another aspect, the present invention provides compounds of formula (I)
or tautomers, or stereoisomers, or pharmaceutically acceptable salts thereof, wherein
R¹, R², R³, and R⁴ are independently selected from H, C₁-C₈-alkyl, and C₁-C₈-alkyl-carboxy;
L is selected from

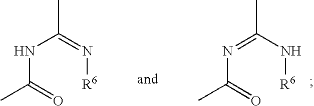

R⁵ and R⁶ are selected from H and C₁-C₈ alkyl;
W is selected from C₁-C₇ alkylene;
X is selected from —NR⁷(C═O)—,
   —NR⁷(C═O)NR⁷—,
   —NR⁸SO₂—,
   —NR⁸(SO₂)NR⁸,
   —NR⁷(C═O)O—,
   —O(C═O)—,
   —O(C═O)O—,
   —O(C═O)NR⁷,
   —(C═O)NR⁷—,
   —(C═O)O—,
   —(SO₂)NR¹⁸—, and
   —(SO₂)NR⁸-Z-(SO₂)NR⁸—;
Y is selected from —C₀-C₈ alkylene- or —(C₀-C₈-alkylene)-SO₂NH—;
Z is C₁ C₄alkylene;

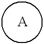

is independently selected from a C₆-C₁₅-membered aromatic carbocyclic group and a 3- to 14-membered heterocyclic group;

R⁷, R⁸, R¹¹ and R¹², are independently selected from H, C₁-C₈-alkyl, C₁-C₈-haloalkyl, a 5- to 14-membered heterocyclic group, and R⁷ and R⁸, independently, by way of an C₁ to C₄ alkyl group can form a bond with a carbon atom of group W or Y creating a 5- to 14-membered heterocyclic group;
T is selected from H, halogen, C₁-C₈ alkyl, C₁-C₈-haloalkyl, C₁-C₈-haloalkoxy, C₃-C₁₅-carbocyclic group, nitro, cyano, a C₆-C₁₅-membered aromatic carbocyclic group, and a C₁-C₈-alkyl substituted by a C₆-C₁₅-membered aromatic carbocyclic group;.
wherein each C₆-C₁₅-membered aromatic carbocyclic group and each 4 to 14 membered heterocyclic group, unless otherwise specified is independently optionally substituted by one or more groups selected from OH, C₁-C₈-alkoxy, C₁-C₈-alkyl, halogen, SO₂NR¹¹R¹², hydroxyC₁-C₈-alkoxy, optionally substituted by hydroxyl, (C₀₋₄alkylene) CONR¹¹R¹², (C₀₋₄alkylene) N═C(NR¹¹R¹²)₂, —O—(C₁₋₄alkylene)-N═C(NR¹¹R¹²)₂, —O—(C₁₋₄alkylene)-CONR¹¹R¹², C₆-C₁₀-aralkoxy, C₇-C₁₀-aralkyl, SH, S(C₁₋₈alkylene), SO₂ (C₁₋₈alkylene) SO(C₁₋₈alkylene), NR¹¹R¹², R¹⁵, a C₁-C₈-alkyl substituted by R¹⁵, R¹⁶, a C₁-C₈-alkyl substituted by R¹⁶, O(C₁-C₈-alkylene)-NR¹¹C(C═O)O—(C₀-C₄-alkylene)-R¹⁵, cyano, oxo, carboxy, nitro, C₁-C₈-alkylcarbonyl, hydroxy-C₁-C₈-alkyl, C₁-C₈-haloalkyl, amino-C₁-C₈-alkyl, amino(hydroxy)C₁-C₈-alkyl and C₁-C₈-alkoxy optionally substituted by aminocarbonyl;
and wherein each alkylene group, unless otherwise specified, is optionally substituted by C₁-C₈-alkyl, halogen, C₁-C₈-alkoxy, carboxy, C₁-C₈-alkyl-carboxy, C₁-C₈-haloalkyl, C₁-C₈-haloalkoxy, C₃-C₁₅-carbocyclic group, C₁-C₈-alkylcarbonyl, C₁-C₈-alkoxycarbonyl, nitro, cyano, R¹⁵, a C₁-C₈-alkyl substituted by R¹⁵, R¹⁶ or a C₁-C₈-alkyl substituted by R¹⁶;
R¹⁵ is a C₆-C₁₅-membered aromatic carbocyclic group, optionally substituted by OH, C₁-C₈-alkoxy, C₁-C₈-alkyl, halogen and C₁-C₈-haloalkyl; and
R¹⁶ is a 3 to 14 membered heterocyclic group, optionally substituted by OH, C₁-C₈-alkoxy, C₁-C₈-alkyl, halogen and C₁-C₈-haloalkyl.

In compounds of formula (I), the following meanings are preferred independently, collectively or in any combination:

According to formula (I), L is suitably

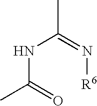

Equally suitably, L is

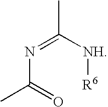

According to formula (I), R¹ is preferably H.

According to formula (I), R² is preferably H.

According to formula (I), R³ is preferably H.

According to formula (I), R⁴ is preferably H.

According to formula (I), R⁵ is preferably H.

According to formula (I), R⁶ is preferably H.

According to formula (I), where A is an optionally substituted 6- to 14-membered aromatic carbocyclic group, this is suitably a phenyl or naphthyl group, preferably phenyl.

According to formula (I), where A is a 4- to 14-membered heterocyclic group, this is suitably a 5 or 6 membered non-aromatic group containing one nitrogen, e.g. a 2-oxo-pyrrolidinyl, e.g. 2-oxo-pyrrolidin-3-yl, a bridged bicylic group containing one nitrogen, e.g. (1S,3S,5R) 8 benzyl-8-aza-bicyclo[3.2.1]oct-3-ylamine or tricyclic group containing one nitrogen, e.g. dibenzoazepine optionally substituted by C₇-aralkyl.

According to formula (I), where A is phenyl, the phenyl is optionally substituted by one or more, preferably one to three, groups independently selected from OH, C₁-C₄ alkyl, e.g. methyl, ethyl or t-butyl, halogen, e.g. chloro or fluoro, C₁-C₄ alkoxy, e.g. methoxy or ethoxy, SO₂NR¹¹R¹², e.g. ethylaminosulfonyl, O—C₁-C₁₀-aralkyl, e.g. benzyloxy, or O(C₀-C₈-alkylene)-NR¹¹C(C═O)O—(C₀-C₄-alkylene)-C₆-C₁₅-membered aromatic carbocyclic group, e.g. 3-propoxy-carbamic acid benzyl ester.

According to formula (I), where A is naphthyl, the naphthyl is optionally substituted by one or more, preferably one group selected from amino and halogen.

According to formula (I), W is suitably methylene, ethylene, butylene, pentylene or hexylene optionally substituted by C₁-C₄ alkyl, e.g. isobutyl, C₁-C₄alkoxycarbonyl, e.g. ethoxycarbonyl, or a 5-14 membered heterocyclic, e.g. indolyl, e.g. 3-indolyl. Preferably, W is C₂-C₆ alkyl.

According to formula (I), X is suitably —NR⁷(C═O)—, —NR⁷(C═O)NR⁷—, —NR⁸SO₂—, —NR⁸(SO₂)NR⁸—, —NR⁷(C═O)O—, —O(C═O)—, —O(C═O)O—, —O(C═O)NR⁷—, —(C═O)NR⁷—, —(C═O)O—, —(SO₂)NR⁸—, —(SO₂)NR⁸(CR⁹R¹⁰)ₙ(SO₂)NR⁸—, or —(C═O)NR⁷—
wherein R⁷ forms a bond with W to provide:

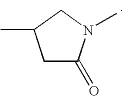

Preferably X is —NHC(═O)NH—, —NHC(═O)—, —NHSO₂—, —SO₂NH—, —C(═O)NH—, —C(═O)N(C₇— aralkyl)-, or

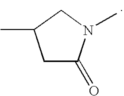

According to formula (I), Y is suitably —(C₀-C₂-alkylene)- or —(C₀-C₂-alkylene)-SO₂NH—. Preferably Y is C₀, i.e. a bond, methylene, ethylene, or —CH₂SO₂NH—.

According to formula (I), W and Y together suitably form a chain length of between two to six atoms.

According to formula (I), T is suitably halogen, preferably chlorine.

In another embodiment, the present invention provides for the use of a compound of formula (I) in any of the aforementioned embodiments, in free or pharmaceutically acceptable salt form, for the manufacture of a medicament for the treatment of an inflammatory or allergic condition, particularly an inflammatory or obstructive airways disease.

A preferred embodiment of the present invention provides for the use of a compound of formula (I) in any of the aforementioned embodiments, in free or pharmaceutically acceptable salt form, for the manufacture of a medicament for the treatment of an inflammatory or allergic condition selected from cystic fibrosis, primary ciliary dyskinesia, chronic bronchitis, chronic obstructive pulmonary disease, asthma, respiratory tract infections, lung carcinoma, xerostomia, and keratoconjunctivitis sire.

It is understood that any and all embodiments of the present invention may be taken in conjunction with any other embodiment to describe additional embodiments of the present invention. Furthermore, any elements of an embodiment are meant to be combined with any and all other elements from any of the embodiments to describe additional embodiments. It is understood by those skilled in the art that combinations of substituents where not possible are not an aspect of the present invention.

### DEFINITIONS

Terms used in the specification have the following meanings:

“Optionally substituted” means the group referred to can be substituted at one or more positions by any one or any combination of the radicals listed thereafter.

“Halo” or “halogen”, as used herein, may be fluorine, chlorine, bromine or iodine.

“C₁-C₈-Alkyl”, as used herein, denotes straight chain or branched alkyl having 1-8 carbon atoms.

“C₁-C₈-Alkoxy”, as used herein, denotes straight chain or branched alkoxy having 1-8 carbon atoms.

The term ‘alkylene’ denotes a straight chain or branched saturated hydrocarbon chain.

“Amino-C₁-C₈-alkyl” and “amino-C₁-C₈-alkoxy” denote amino attached by a nitrogen atom to C₁-C₈-alkyl, e.g., NH₂—(C₁-C₈)—, or to C₁-C₈-alkoxy, e.g., NH₂—(C₁-C₈)—O—. “Amino-(hydroxy)-C₁-C₈-alkyl” denotes amino attached by a nitrogen atom to C₁-C₈-alkyl and hydroxy attached by an oxygen atom to the same C₁-C₈-alkyl.

“C₁-C₈-Alkylcarbonyl” and “C₁-C₈-alkoxycarbonyl”, as used herein, denote C₁-C₈-alkyl or C₁-C₈-alkoxy, respectively, as hereinbefore defined, attached by a carbon atom to a carbonyl group.

“C₃-C₈-Cycloalkylcarbonyl”, as used herein, denotes C₃-C₈-cycloalkyl, as hereinbefore defined, attached by a carbon atom to a carbonyl group.

“C₇-C₁₄-Aralkyl”, as used herein, denotes alkyl, e.g., C₁-C₄-alkyl, as hereinbefore defined, substituted by a C₆-C₁₀-aromatic carbocyclic group, as herein defined.

Aryl equivalent to “C₆-C₁₅-Aromatic carbocyclic group”

“C₃-C₁₅-carbocyclic group”, as used herein, denotes a carbocyclic group having 3- to 15-ring carbon atoms that is saturated or partially saturated, such as a C₃-C₈-cycloalkyl. Examples of C₃-C₁₅-carbocyclic groups include but are not limited to cyclopropyl, cyclobutyl, cyclopentyl, cyclohexyl, cycloheptyl or cyclooctyl or a bicyclic group, such as bicyclooctyl, bicyclononyl including indanyl and indenyl, and bicyclodecyl.

“C₆-C₁₅-aromatic carbocyclic group”, as used herein, denotes an aromatic group having 6- to 15-ring carbon atoms. Examples of C₆-C₁₅-Aromatic carbocyclic groups include but are not limited to phenyl, phenylene, benzenetriyl, naphthyl, naphthylene, naphthalenetriyl or anthrylene.

“3- to 14-membered heterocyclic group” refers to a 3- to 14-membered heterocyclic ring containing at least one ring heteroatom selected from the group consisting of nitrogen, oxygen and sulphur, which may be saturated, partially saturated or unsaturated (aromatic). Examples of 3- to 14-membered heterocyclic groups include but are not limited to furan, pyrrole, pyrrolidine, pyrazole, imidazole, triazole, isotriazole, tetrazole, thiadiazole, isothiazole, oxadiazole, pyridine, piperidine, pyrazine, oxazole, isoxazole, pyrazine, pyridazine, pyrimidine, piperazine, pyrrolidine, pyrrolidinone, morpholine, triazine, oxazine, tetrahyrofuran, tetrahydrothiophene, tetrahydrothiopyran, tetrahydropyran, 1,4-dioxane, 1,4-oxathiane, indazole, quinoline, indazole, indole or thiazole.

Throughout this specification and in the claims that follow, unless the context requires otherwise, the word “comprise”, or variations, such as “comprises” or “comprising”, will be understood to imply the inclusion of a stated integer or step or group of integers or steps but not the exclusion of any other integer or step or group of integers or steps.

Especially preferred specific compounds of formula (I) are those described hereinafter in the Examples.

The compounds represented by formula (I) may be capable of forming acid addition salts, particularly pharmaceutically acceptable acid addition salts. Pharmaceutically acceptable acid addition salts of the compound of formula (I) include those of inorganic acids, e.g., hydrohalic acids, such as hydrofluoric acid, hydrochloric acid, hydrobromic acid or hydroiodic acid, nitric acid, sulfuric acid, phosphoric acid; and organic acids, e.g., aliphatic monocarboxylic acids, such as formic acid, acetic acid, trifluoroacetic acid, propionic acid and butyric acid; aliphatic hydroxy acids, such as lactic acid, citric acid, tartaric acid or malic acid; dicarboxylic acids, such as maleic acid or succinic acid; aromatic carboxylic acids, such as benzoic acid, p-chlorobenzoic acid, diphenylacetic acid, para-biphenyl benzoic acid or triphenylacetic acid; aromatic hydroxy acids, such as o-hydroxybenzoic acid, p-hydroxybenzoic acid, 1-hydroxynaphthalene-2-carboxylic acid or 3-hydroxynaphthalene-2-carboxylic acid; cinnamic acids, such as 3-(2-naphthalenyl)propenoic acid, para-methoxy cinnamic acid or para-methyl cinnamic acid; and sulfonic acids, such as methanesulfonic acid or benzenesulfonic acid. These salts may be prepared from compounds of formula (I) by known salt-forming procedures.

Compounds of formula (I) which may contain acidic, e.g., carboxyl, groups, are also capable of forming salts with bases, in particular, pharmaceutically acceptable bases, such as those well-known in the art; suitable such salts include metal salts, particularly alkali metal or alkaline earth metal salts, such as sodium, potassium, magnesium or calcium salts; or salts with ammonia or pharmaceutically acceptable organic amines or heterocyclic bases, such as ethanolamines, benzylamines or pyridine. These salts may be prepared from compounds of formula (I) by known salt-forming procedures.

Stereoisomers are those compounds where there is an asymmetric carbon atom. The compounds exist in individual optically active isomeric forms or as mixtures thereof, e.g., as diastereomeric mixtures. The present invention embraces both individual optically active R and S isomers, as well as mixtures thereof. Individual isomers can be separated by methods well known to those skilled in the art, e.g. chiral high performance liquid chromatography (HPLC).

Tautomers are one of two or more structural isomers that exist in equilibrium and are readily converted from one isomeric form to another.

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

### Synthesis

An embodiment of the present invention provides a process for the preparation of compounds of formula (I), or tautomers, or stereoisomers, or pharmaceutically acceptable salts thereof,

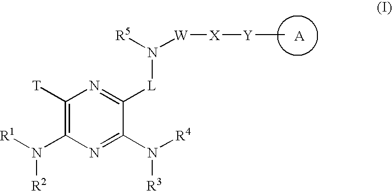

wherein R¹, R², R³, R⁴, R⁵, T, L, W, X, Y, and

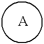

are as defined hereinbefore, which comprises the steps of:
(i) reacting a compound of formula (IV)

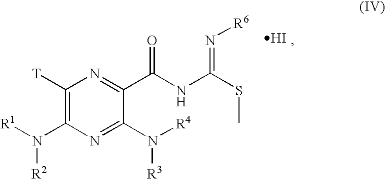

wherein R¹, R², R³, R⁴, R⁶ and T are as hereinbefore defined,
with compounds of formula (V)

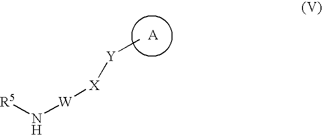

wherein R⁵, W, X, Y, and

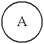

are hereinbefore defined, optionally in the presence of a base, e.g., an organic base; and in an organic solvent, e.g., a non-protic dipolar solvent; and
(ii) recovering the resultant compound of formula (I), in free or pharmaceutically acceptable salt form.

The compounds of formula (I) can be prepared, e.g., using the reactions and techniques described below and in the Examples. The reactions may be performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the invention.

The various substituents on the synthetic intermediates and final products shown in the following reaction schemes can be present in their fully elaborated forms, with suitable protecting groups where required as understood by one skilled in the art, or in precursor forms which can later be elaborated into their final forms by methods familiar to one skilled in the art. The substituents can also be added at various stages throughout the synthetic sequence or after completion of the synthetic sequence. In many cases, commonly used functional group manipulations can be used to transform one intermediate into another intermediate, or one compound of formula (I) into another compound of formula (I). Examples of such manipulations are conversion of an ester or a ketone to an alcohol; conversion of an ester to a ketone; interconversions of esters, acids and amides; alkylation, acylation and sulfonylation of alcohols and amines; and many others. Substituents can also be added using common reactions, such as alkylation, acylation, halogenation or oxidation. Such manipulations are well-known in the art, and many reference works summarize procedures and methods for such manipulations. Some reference works which gives examples and references to the primary literature of organic synthesis for many functional group manipulations, as well as other transformations commonly used in the art of organic synthesis are March's Organic Chemistry, 5^{th} Edition, Wiley and Chichester, Eds. (2001); Comprehensive Organic Transformations, Larock, Ed., VCH (1989); Comprehensive Organic Functional Group Transformations, Katritzky et al. (series editors), Pergamon (1995); and Comprehensive Organic Synthesis, Trost and Fleming (series editors), Pergamon (1991). It will also be recognized that another major consideration in the planning of any synthetic route in this field is the judicious choice of the protecting group used for protection of the reactive functional groups present in the compounds described in this invention. Multiple protecting groups within the same molecule can be chosen such that each of these protecting groups can either be removed without removal of other protecting groups in the same molecule, or several protecting groups can be removed using the same reaction step, depending upon the outcome desired. An authoritative account describing many alternatives to the trained practioner is Greene and Wuts, Protective Groups in Organic Synthesis, Wiley and Sons (1999).

Generally, compounds described in the scope of this patent application can be synthesized by the routes described in Scheme 1 and the Examples.

In Scheme 1, compounds of formula (I) can be prepared according to the processes described by Cragoe et al., J Med Chem, Vol. 10, pp. 66-73 (1967); and European Patent EP 0 017 152 and US patent U.S. Pat. No. 3,544,571. For instance, intermediate 1 can be reacted with intermediate 2 in the presence of triethylamine in organic solvent to provide compound 3 as the free base. The free base can then be converted to a salt form by treatment with an appropriate acid. Intermediates can be prepared from methods known by those skilled in the art or are commercially available.

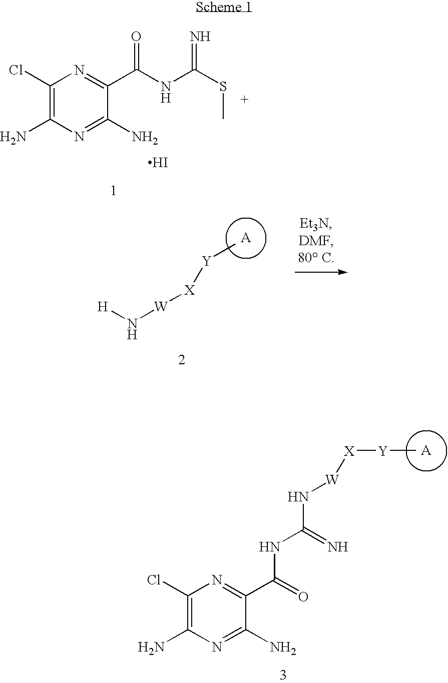

Compounds of formula (I), in free form, may be converted into salt form, and vice versa, in a conventional manners understood by those skilled in the art. The compounds in free or salt form can be obtained in the form of hydrates or solvates containing a solvent used for crystallisation. Compounds of formula (I) can be recovered from reaction mixtures and purified in a conventional manner. Isomers, such as stereoisomers, may be obtained in a conventional manner, e.g., by fractional crystallisation or asymmetric synthesis from correspondingly asymmetrically substituted, e.g., optically active, starting materials.

### Pharmacological Activity

Having regard to their blockade of the epithelial sodium channel (ENaC), compounds of formula (I), in free or pharmaceutically acceptable salt form, hereinafter alternately referred to as “agents of the invention”, are useful in the treatment of conditions which respond to the blockade of the epithelial sodium channel, particularly conditions benefiting from mucosal hydration.

Diseases mediated by blockade of the epithelial sodium channel, include diseases associated with the regulation of fluid volumes across epithelial membranes. For example, the volume of airway surface liquid is a key regulator of mucociliary clearance and the maintenance of lung health. The blockade of the epithelial sodium channel will promote fluid accumulation on the mucosal side of the airway epithelium thereby promoting mucus clearance and preventing the accumulation of mucus and sputum in respiratory tissues (including lung airways). Such diseases include respiratory diseases, such as cystic fibrosis, primary ciliary dyskinesia, chronic bronchitis, chronic obstructive pulmonary disease (COPD), asthma, respiratory tract infections (acute and chronic; viral and bacterial) and lung carcinoma. Diseases mediated by blockade of the epithelial sodium channel also include diseases other than respiratory diseases that are associated with abnormal fluid regulation across an epithelium, perhaps involving abnormal physiology of the protective surface liquids on their surface, e.g., xerostomia (dry mouth) or keratoconjunctivitis sire (dry eye). Furthermore, blockade of the epithelial sodium channel in the kidney could be used to promote diuresis and thereby induce a hypotensive effect.

Treatment in accordance with the invention may be symptomatic or prophylactic.

Asthma includes both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g., of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g., of acute asthmatic or bronchoconstrictor attack, improvement in lung function or improved airways hyperreactivity. It may further be evidenced by reduced requirement for other, symptomatic therapy, i.e., therapy for or intended to restrict or abort symptomatic attack when it occurs, e.g., anti-inflammatory (e.g., cortico-steroid) or bronchodilatory. Prophylactic benefit in asthma may, in particular, be apparent in subjects prone to “morning dipping”. “Morning dipping” is a recognized asthmatic syndrome, common to a substantial percentage of asthmatics and characterized by asthma attack, e.g., between the hours of about 4-6 am, i.e., at a time normally substantially distant from any previously administered symptomatic asthma therapy.

Chronic obstructive pulmonary disease includes chronic bronchitis or dyspnea associated therewith, emphysema, as well as exacerbation of airways hyperreactivity consequent to other drug therapy, in particular, other inhaled drug therapy. The invention is also applicable to the treatment of bronchitis of whatever type or genesis including, e.g., acute, arachidic, catarrhal, croupus, chronic or phthinoid bronchitis.

The suitability of epithelial sodium channel blocker as a treatment of a disease benefiting from mucosal hydration, may be tested by determining the inhibitory effect of the channel activating protease inhibitor on: the ion channel/ion transport function in suitable isolated cells or confluent epithelia using the methods described in Bridges et al., Am J Physiol Lung Cell Mol Physiol, Vol. 281, No. 1, pp. L16-L23 (2001); and Donaldson et al., J Biol Chem, Vol. 277, No. 10, pp. 8338-8345 (2002).

Epithelial sodium channel blockers, including the compounds of formula (I), are also useful as co-therapeutic agents for use in combination with other drug substances, such as anti-inflammatory, bronchodilatory, antihistamine or anti-tussive drug substances, particularly in the treatment of cystic fibrosis or obstructive or inflammatory airways diseases such as those mentioned hereinbefore, e.g., as potentiators of therapeutic activity of such drugs or as a means of reducing required dosaging or potential side effects of such drugs.

The epithelial sodium channel blocker may be mixed with the other drug substance in a fixed pharmaceutical composition or it may be administered separately, before, simultaneously with or after the other drug substance.

Accordingly, the invention includes a combination of epithelial sodium channel blocker with an anti-inflammatory, bronchodilatory, antihistamine, anti-tussive, antibiotic or DNase drug substance, said epithelial sodium channel blocker and said drug substance being in the same or different pharmaceutical composition.

Suitable antibiotics include macrolide antibiotics, e.g., tobramycin (TOBI™).

Suitable DNase drug substances include dornase alfa (Pulmozyme™), a highly-purified solution of recombinant human deoxyribonuclease I (rhDNase), which selectively cleaves DNA. Dornase alfa is used to treat cystic fibrosis.

Other useful combinations of epithelial sodium channel blockers with anti-inflammatory drugs are those with antagonists of chemokine receptors, e.g., CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists, such as Schering-Plough antagonists SC-351125, SCH-55700 and SCH-D; Takeda antagonists, such as N-[[4-[[[6,7-dihydro-2-(4-methyl-phenyl)-5H-benzo-cyclohepten-8-yl]carbonyl]amino]phenyl]-methyl]tetrahydro-N,N-dimethyl-2H-pyran-4-amin-ium chloride (TAK-770); and CCR-5 antagonists described in U.S. Pat. No. 6,166,037 (particularly claims 18 and 19), WO 00/66558 (particularly claim 8), WO 00/66559 (particularly claim 9), WO 04/018425 and WO 04/026873.

Suitable anti-inflammatory drugs include steroids, in particular, glucocorticosteroids, such as budesonide, beclamethasone dipropionate, fluticasone propionate, ciclesonide or mometasone furoate, or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/35668, WO 03/48181, WO 03/62259, WO 03/64445, WO 03/72592, WO 04/39827 and WO 04/66920; non-steroidal glucocorticoid receptor agonists, such as those described in DE 10261874, WO 00/00531, WO 02/10143, WO 03/82280, WO 03/82787, WO 03/86294, WO 03/104195, WO 03/101932, WO 04/05229, WO 04/18429, WO 04/19935 and WO 04/26248; LTD4 antagonists, such as montelukast and zafirlukast; PDE4 inhibitors, such as cilomilast (Ariflo® GlaxoSmithKline), Roflumilast (Byk Guiden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SelCID™ CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; adenosine A2B receptor antagonists such as those described in WO 02/42298; and beta-2 adrenoceptor agonists, such as albuterol (salbutamol), metaproterenol, terbutaline, salmeterol fenoterol, procaterol, and especially, formoterol, carmoterol and pharmaceutically acceptable salts thereof, and compounds (in free or salt or solvate form) of formula (I) of WO 0075114, which document is incorporated herein by reference, preferably compounds of the Examples thereof, especially a compound of formula

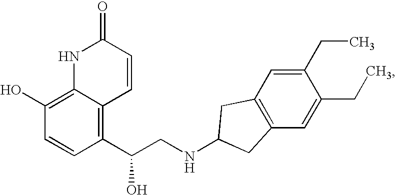

corresponding to indacaterol and pharmaceutically acceptable salts thereof, as well as compounds (in free or salt or solvate form) of formula (I) of WO 04/16601, and also compounds of EP 1440966, JP 05025045, WO 93/18007, WO 99/64035, USP 2002/0055651, WO 01/42193, WO 01/83462, WO 02/66422, WO 02/70490, WO 02/76933, WO 03/24439, WO 03/42160, WO 03/42164, WO 03/72539, WO 03/91204, WO 03/99764, WO 04/16578, WO 04/22547, WO 04/32921, WO 04/33412, WO 04/37768, WO 04/37773, WO 04/37807, WO 04/39762, WO 04/39766, WO 04/45618, WO 04/46083, WO 04/80964, WO 04/108765 and WO 04/108676.

Suitable bronchodilatory drugs include anticholinergic or antimuscarinic agents, in particular, ipratropium bromide, oxitropium bromide, tiotropium salts and CHF 4226 (Chiesi), and glycopyrrolate, but also those described in EP 424021, U.S. Pat. No. 3,714,357, U.S. Pat. No. 5,171,744, WO 01/04118, WO 02/00652, WO 02/51841, WO 02/53564, WO 03/00840, WO 03/33495, WO 03/53966, WO 03/87094, WO 04/018422 and WO 04/05285.

Suitable dual anti-inflammatory and bronchodilatory drugs include dual beta-2 adrenoceptor agonist/muscarinic antagonists such as those disclosed in USP 2004/0167167, WO 04/74246 and WO 04/74812.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine, as well as those disclosed in JP 2004107299, WO 03/099807 and WO 04/026841.

Other useful combinations of agents of the invention with anti-inflammatory drugs are those with antagonists of chemokine receptors, e.g., CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists, such as Schering-Plough antagonists SC-351125, SCH-55700 and SCH-D; Takeda antagonists, such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-amin-ium chloride (TAK-770), and CCR-5 antagonists described in U.S. Pat. No. 6,166,037 (particularly claims 18 and 19), WO 00/66558 (particularly claim 8), WO 00/66559 (particularly claim 9), WO 04/018425 and WO 04/026873.

In accordance with the foregoing, the invention also provides a method for the treatment of a condition responsive to blockade of the epithelial sodium channel, e.g., diseases associated with the regulation of fluid volumes across epithelial membranes, particularly an obstructive airways disease, which comprises administering to a subject, particularly a human subject, in need thereof a compound of formula (I), in free form or in the form of a pharmaceutically acceptable salt. In another aspect the invention provides a compound of formula (I), in free form or in the form of a pharmaceutically acceptable salt, for use in the manufacture of a medicament for the treatment of a condition responsive to blockade of the epithelial sodium channel, particularly an obstructive airways disease, e.g., Cystic Fibrosis and COPD.

The agents of the invention may be administered by any appropriate route, e.g. orally, e.g., in the form of a tablet or capsule; parenterally, e.g., intravenously; by inhalation, e.g., in the treatment of an obstructive airways disease; intranasally, e.g., in the treatment of allergic rhinitis; topically to the skin; or rectally. In a further aspect, the invention also provides a pharmaceutical composition comprising a compound of formula (I), in free form or in the form of a pharmaceutically acceptable salt, optionally together with a pharmaceutically acceptable diluent or carrier therefor. The composition may contain a co-therapeutic agent, such as an anti-inflammatory, broncho-dilatory, antihistamine or anti-tussive drug as hereinbefore described. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g., patches. Compositions for inhalation may comprise aerosol or other atomizable formulations or dry powder formulations.

When the composition comprises an aerosol formulation, it preferably contains, e.g., a hydro-fluoro-alkane (HFA) propellant, such as HFA134a or HFA227 or a mixture of these, and may contain one or more co-solvents known in the art, such as ethanol (up to 20% by weight), and/or one or more surfactants, such as oleic acid or sorbitan trioleate, and/or one or more bulking agents, such as lactose. When the composition comprises a dry powder formulation, it preferably contains, e.g., the compound of formula (I) having a particle diameter up to 10 microns, optionally together with a diluent or carrier, such as lactose, of the desired particle size distribution and a compound that helps to protect against product performance deterioration due to moisture, e.g., magnesium stearate. When the composition comprises a nebulised formulation, it preferably contains, e.g., the compound of formula (I) either dissolved, or suspended, in a vehicle containing water, a co-solvent, such as ethanol or propylene glycol and a stabilizer, which may be a surfactant.

The invention includes:
(a) a compound of formula (I) in inhalable form, e.g., in an aerosol or other atomisable composition or in inhalable particulate, e.g., micronised form;
(b) an inhalable medicament comprising a compound of formula (I) in inhalable form;
(c) a pharmaceutical product comprising a compound of formula (I) in inhalable form in association with an inhalation device; and
(d) an inhalation device containing a compound of formula I in inhalable form.

Dosages of compounds of formula (I) employed in practising the present invention will of course vary depending, e.g., on the particular condition to be treated, the effect desired and the mode of administration. In general, suitable daily dosages for administration by inhalation are of the order of 0.005-10 mg, while for oral administration suitable daily doses are of the order of 0.05-100 mg.

### Pharmaceutical Use and Assay

Compounds of formula (I), (II) and (III) and their pharmaceutically acceptable salts, hereinafter referred to alternatively as “agents of the invention”, are useful as pharmaceuticals. In particular, the compounds have good ENaC blocker activity and may be tested in the following assays.

### Cell Culture

Human Bronchial Epithelial cells (HBECs) (Cambrex) were cultured under air-liquid interface conditions to provide a well differentiated mucociliary phenotype.

HBECs were cultured using a modification of the method described by Gray and colleagues

(Gray et al., 1996). Cells were seeded in plastic T-162 flasks and were grown in bronchial epithelial cell growth medium (BEGM; Cambrex) supplemented with bovine pituitary extract (52 μg/mL), hydrocortisone (0.5 μg/mL), human recombinant epidermal growth factor (0.5 μg/mL), epinephrine (0.5 μg/mL), transferrin (10 μg/mL), insulin (5 μg/mL), retinoic acid (0.1 μg/mL), triiodothyronine (6.5 μg/mL), gentamycin (50 μg/mL) and amphotericin B (50 μg/mL). Medium was changed every 48 hours until cells were 90% confluent. Cells were then passaged and seeded (8.25×10⁵ cells/insert) on polycarbonate Snapwell inserts (Costar) in differentiation media containing 50% DMEM in BEGM with the same supplements as above but without triiodothyronine and a final retinoic acid concentration of 50 nM (all-trans retinoic acid). Cells were maintained submerged for the first 7 days in culture, after which time they were exposed to an apical air interface for the remainder of the culture period. At this time, media was changed to DMEM:F12 media containing 2% v/v Ultroser G for the remainder of culture. Amphotericin B was removed from all media 3 feeds prior to use in the Ussing Chambers. Cells were used between days 7 and 21 after establishment of the apical-air interface. At all stages of culture, cells were maintained at 37° C. in 5% CO₂ in an air incubator.

### Short Circuit Current (ISC) Measurements

Snapwell inserts were mounted in Vertical Diffusion Chambers (Costar) and were bathed with continuously gassed Ringer solution (5% CO₂ in O₂; pH 7.4) maintained at 37° C. containing (in mM): 120 NaCl, 25 NaHCO₃, 3.3 KH₂PO₄, 0.8 K₂HPO₄, 1.2 CaCl₂, 1.2 MgCl₂, and 10 glucose. The solution osmolarity was between 280 and 300 mOsmol/kg H₂O for all physiological salt solutions used. Cells were voltage clamped to 0 mV (model EVC4000; WPI). RT was measured by applying a 1- or 2-mV pulse at 30-s intervals and calculating RT by Ohm's law. Data were recorded using a PowerLab workstation (ADInstruments).

Test compounds were prepared as a 10 mM stock solution in DMSO (95%). Serial 3-fold dilutions were freshly prepared in an appropriate vehicle (distilled H₂O or Ringers solution). The initial concentration was added to the apical chamber as a 1000× concentrate in 5 μL, resulting in a final 1× concentration the 5 mL volume of the Ussing chamber. Subsequent additions of compound were added in a 3.3 μL volume of the 1000× serially diluted stock solution. At the completion of the concentration-response experiment, amiloride (10 μM) was added into the apical chamber to enable the total amiloride-sensitive current to be measured. An amiloride control IC₅₀ was established at the start of each experiment.

Results are expressed as the mean % inhibition of the amiloride-sensitive ISC. Concentration-response curves were plotted and IC₅₀ values generated using GraphPad Prism 3.02. Cell inserts were typically run in duplicate and the IC₅₀ calculated on the mean % inhibition data.

Compounds of the Examples, herein below, generally have IC₅₀ values in the data measurements described above below 10 μM. For example, the compounds of Examples 3, 12, 17 and 25 have IC₅₀ values of 0.01645, 0.06585, 0.033 and 0.018 μM, respectively.

The invention is illustrated by the following Examples.

### EXAMPLES

### General Conditions

LCMS are recorded on an Agilent 1100 LC system with a Waters Xterra MS C18 4.6×100 5 μM column, eluting with 5-95% 10 mM aqueous ammonium bicarbonate in acetonitrile over 2.5 minutes, with negative ion electrospray ionization or 5-95% water+0.1% TFA in acetonitrile with positive ion electrospray ionization. [M+H]+ and [M−H]⁻ refer to monoisotopic molecular weights.

| | |
|---|---|
| DMF | dimethylformamide |
| DMSO | dimethyl sulfoxide |
| Et₃N | triethylamine |
| EtOAc | ethyl acetate |
| HPLC | high performance liquid |
| | chromatography |
| MeOH | methanol |
| RT | room temperature |
| TFA | trifluoroacetic acid |

### Example 1 1-{2-[N′-(3,5-Diamino-6-chloro-pyrazine-2-carbonyl)-guanidino]-ethyl}-3-(4-fluoro-phenyl)-urea triflouroacetate

A suspension of 1-(3,5-diamino-6-chloropyrazinoyl)-2-methyl-2-thioseudourea (0.05 g, 0.13 mmol) in peptide grade DMF (2 mL) containing Et₃N (0.182 mmol, 0.025 mL) is treated with 1-(2-amino-ethyl)-3-(4-fluoro-phenyl)-urea (0.0359 g, 0.182 mmol). The reaction mixture is shaken at RT overnight. The solvent is removed under vacuum and the residue resuspended in DMSO (1 mL). The product is purified by Mass Spec directed preparative HPLC to give the title compound.

Examples 2-13 are prepared by processes similar as that described in Example 1, however Examples 4 and 13 utilize 2 equivalents of triethylamine and 2 equivalents of the corresponding amine.

### Example 14 4-Benzyloxy-N-{2-[N′-(3,5-diamino-6-chloro-pyrazine-2-carbonyl)-guanidino]-ethyl}-benzenesulfonamide trifluoroacetate

A stirred suspension of 1-(3,5-diamino-6-chloropyrazinoyl)-2-methyl-2-thioseudourea (0.047 g, 0.13 mmol) in peptide grade DMF (2 mL) wis treated with Et₃N (0.04 mL, 0.26 mmol). A solution of N-(2-amino-ethyl)-4-benzyloxy-benzenesulfonamide (0.068 g, 0.13 mmol) in peptide grade DMF (2 mL) is added and the reaction mixture heated to 50° C. overnight. The solvent is removed under vacuum and the residue resuspended in DMSO (1 mL). The product is purified by Mass Spec directed preparative HPLC to give the title compound.

Examples 15 and 16 are prepared by processes similar to that described in Example 14.

### Example 17 (S)-6-[N′-(3,5-Diamino-6-chloro-pyrazine-2-carbonyl)-guanidino]-2-(toluene-4 sulfonylamino)-hexanoic acid methyl ester trifluoroacetate

A suspension of 1-(3,5-diamino-6-chloropyrazinoyl)-2-methyl-2-thioseudourea (0.05 g, 0.13 mmol) in peptide grade DMF (4 mL) is treated with Et₃N (0.051 mL, 0.36 mmol). (S)-6-amino-2-(toluene-4-sulfonylamino)-hexanoic acid methyl ester (0.064 g, 0.182 mmol) is added (as a 0.4 M solution in DMF) and the reaction mixture is stirred at RT overnight then to 50° C. for a further 18 hours. The solvent is removed under vacuum and the residue resuspended in DMSO (1 mL). The product is purified by Mass Spec directed preparative HPLC to give the title compound.

Examples 18-20 are prepared by similar processes as that described in Example 17.

### Example 21 (S)-2-[N′-(3,5-Diamino-6-chloro-pyrazine-2-carbonyl)-guanidino]-4-methyl-pentanoic acid naphthalen-2-ylamide trifluoroacetate

A suspension of 1-(3,5-diamino-6-chloropyrazinoyl)-2-methyl-2-thioseudourea (0.05 g, 0.13 mmol) in peptide grade DMF (4 mL) is treated with Et₃N (0.051 mL, 0.36 mmol). (S)-2-amino-4-methyl-pentanoic acid naphthalene-2-ylamide (0.053 g, 0.182 mmol) is added and the reaction mixture stirred at RT overnight followed by a further night at 50° C. Further (S)-2-amino-4-methyl-pentanoic acid naphthalene-2-ylamide (0.053 g, 0.182 mmol) is added (as a 0.4 M solution in DMF) and the reaction mixture heated to 70° C. overnight. The solvent is removed under vacuum and the residue resuspended in DMSO (1 mL). The product is purified by Mass Spec directed preparative HPLC to give the title compound.

Example 22 is prepared by similar processes as that described in Example 21.

### Example 23 N-{2-[N′-(3,5-Diamino-6-chloro-pyrazine-2-carbonyl)-guanidino]-ethyl}-benzenesulfonamide

To a suspension of 1-(3,5diamino-6-chloropyrazinoyl)-2-methyl-2-thioseudourea (100 mg, 0.26 mmol) dissolved in MeOH (1 mL) is (N-(2-amino-ethyl)-benzene sulfonamide hydrochloride) (52 mg, 0.26 mmol) and Et₃N (83 μL, 0.59 mmol). Stirring is continued at RT for 18 hours. The reaction is concentrated in vacuo and the product is purified by flash column chromatography (10% MeOH in EtOAc) to produce the title product as the free base.

### Example 24 N-{3-[N′-(3,5-Diamino-6-chloro-pyrazine-2-carbonyl)-guanidino]-propyl}-2-pheny-acetamide

To a suspension of 1-(3,5diamino-6-chloropyrazinoyl)-2-methyl-2-thioseudourea (100 mg, 0.26 mmol) dissolved in MeOH (1.3 mL) is added (N-(3-amino-propyl)-2-phenyl-acetamide hydrochloride) (99 mg, 0.52 mmol) and Et₃N (146 μL, 1.04 mmol). Stirring is continued at RT for 3 hours. DMF (0.5 mL) is added to aid solution and the reaction stirred for a further hour. The reaction is concentrated in vacuo and the product is purified by flash column chromatography (10% MeOH in EtOAc) to produce the title product as the free base.

Examples 25-30 are prepared by similar processes as that described in Example 24.

### Example 31 N-{2-[N′-(3,5-Diamino-6-chloro-pyrazine-2-carbonyl)-guanidino]-ethyl}-2-phenyl-acetamide trifluoroacetate

To a suspension of 1-(3,5diamino-6-chloropyrazinoyl)-2-methyl-2-thioseudourea (100 mg, 0.26 mmol) dissolved in MeOH (1.3 mL) is added (N-(2-amino-ethyl)-2-phenyl-acetamide hydrochloride) (92 mg, 0.52 mmol) and Et₃N (146 μL, 1.04 mmol). Stirring is continued at RT for 3 hours. DMF (0.5 mL) is added to aid solution and the reaction stirred for a further hour. The reaction is concentrated in vacuo and the product is purified by reverse phase column chromatography (0-100% acetonitrile gradient over 25 minutes and 0.05% TFA modifier in both aqueous and organic phases) to give the title product as the trifluoroacetate salt.

Examples 32 and 33 are prepared by similar processes as that described in Example 31.

### Example 34 N-{2-[N′-(3,5-Diamino-6-chloro-pyrazine-2-carbonyl)-guanidino]-ethyl}-benzamide

To a suspension of 1-(3,5diamino-6-chloropyrazinoyl)-2-methyl-2-thioseudourea (100 mg, 0.26 mmol) dissolved in MeOH (1 mL) is added (N-(2-amino-ethyl)-benzamide hydrochloride) (43 mg, 0.26 mmol) and Et₃N (83 μL, 0.59 mmol). Stirring is continued at RT for 18 hours. The product is filtered to produce the title product as the free base.

The compounds of Examples 1-34, of general structure VI, are prepared using the appropriate starting compounds and methods as outlined above.

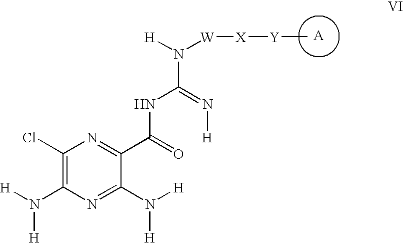

TABLE 1

| | | M/s |
|---|---|---|
| Ex. | Structure | M+ |
| 1 | 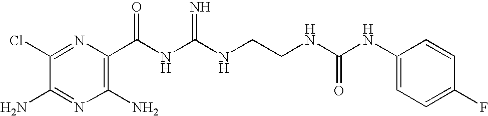 | 410.1 |
| 2 | 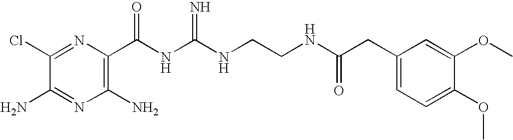 | 451.4 |
| 3 | 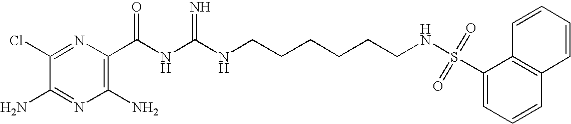 | 519.1 |
| 4 | 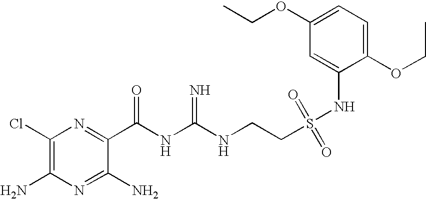 | 501.22 |
| 5 | 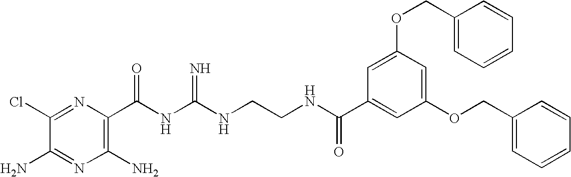 | 589.17 |
| 6 | 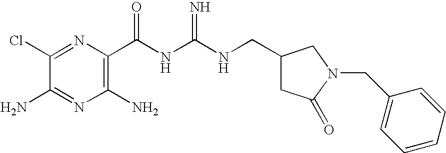 | 417.4 |
| 7 | 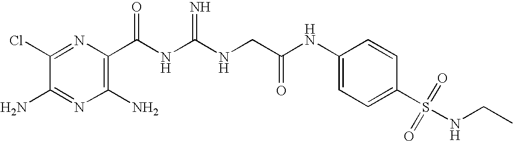 | 470.1 |
| 8 | 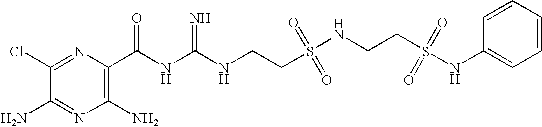 | 520.3 |
| 9 | 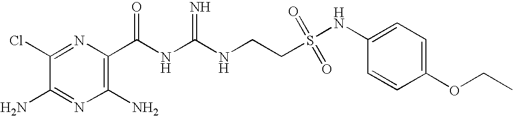 | 457.1 |
| 10 | 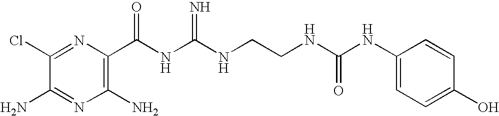 | 408.4 |
| 11 | 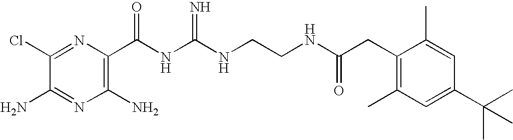 | 475.2 |
| 12 | 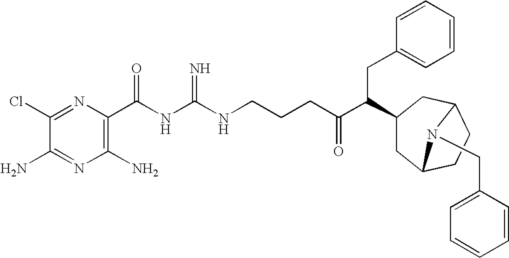 | 604.5 |
| 13 | 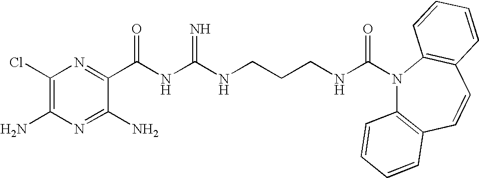 | 506.43 |
| 14 | 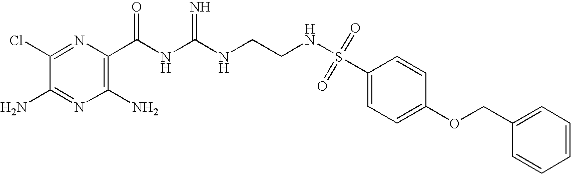 | 519.4 |
| 15 | 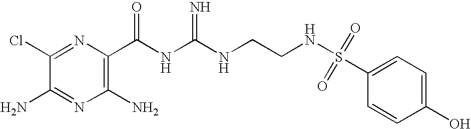 | 429.3 |
| 16 | 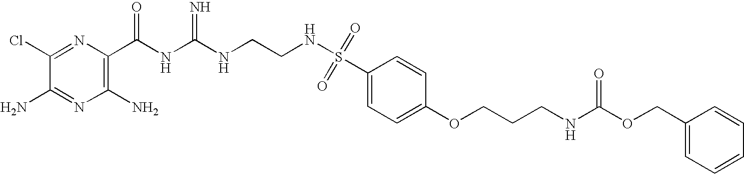 | 620.4 |
| 17 | 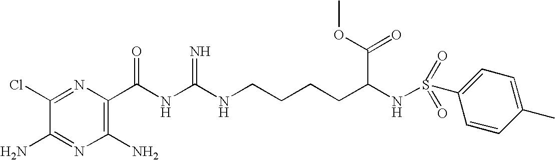 | 527.06 |
| 18 | 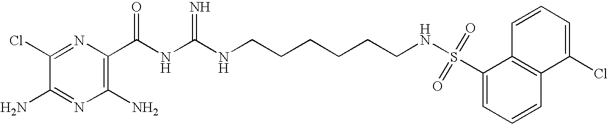 | 533.04 |
| 19 | 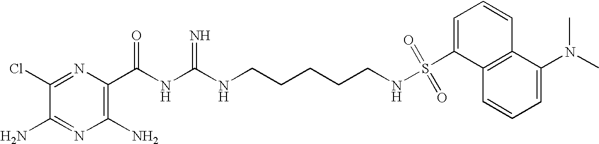 | 548.13 |
| 20 | 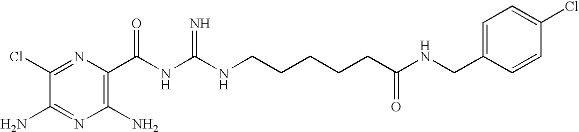 | 467.07 |
| 21 | 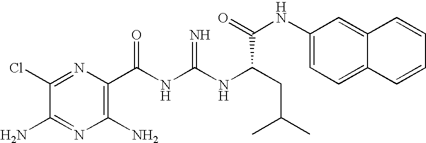 | 469.13 |
| 22 | 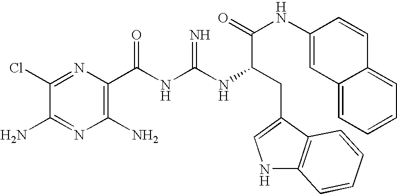 | 542.12 |
| 23 | 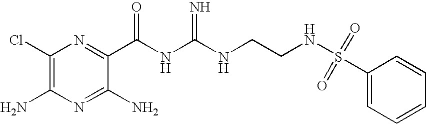 | 413.26 |
| 24 | 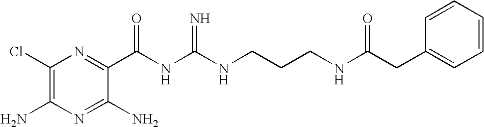 | 405.21 |
| 25 | 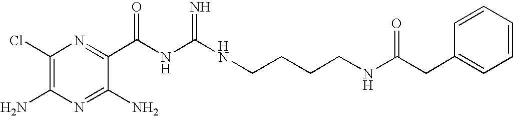 | 419.15 |
| 26 | 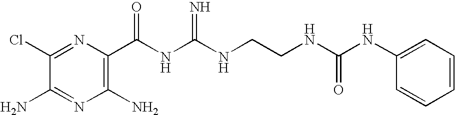 | 392.13 |
| 27 | 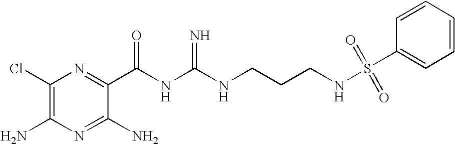 | 427.11 |
| 28 | 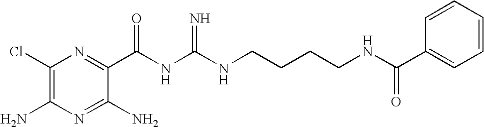 | 405.19 |
| 29 | 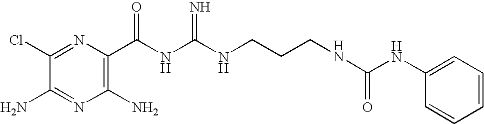 | 406.16 |
| 30 | 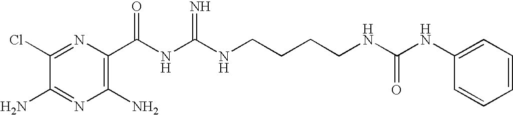 | 420.19 |
| 31 | 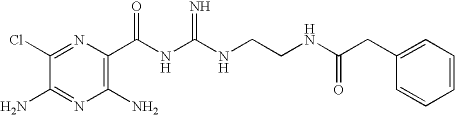 | 391.07 |
| 32 | 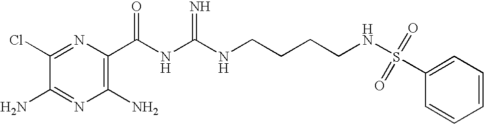 | 441.07 |
| 33 | 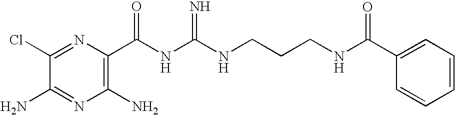 | 391.07 |
| 34 | 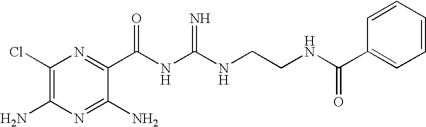 | 377.26 |

1. A compound of formula (I) 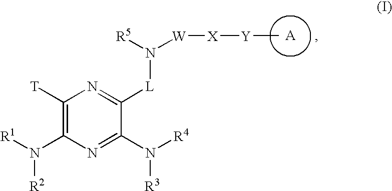
or tautomers, or stereoisomers, or solvates, or pharmaceutically acceptable salts thereof, wherein
R¹, R², R³, and R⁴ are independently selected from H, C₁-C₈-alkyl, C₁-C₈-alkyl-carboxy, C₁-C₈-haloalkyl, C₃-C₁₅-carbocyclic group, C₁-C₈-alkylcarbonyl, C₁-C₈-alkoxycarbonyl, a C₆-C₁₅-membered aromatic carbocyclic group, a 3- to 14-membered heterocyclic group, a C₁-C₈-alkyl substituted by a 3- to 14-membered heterocyclic group, and a C₁-C₈-alkyl substituted by a C₆-C₁₅-membered aromatic carbocyclic group,
or R¹ and R² with the nitrogen atom to which they are attached form a C₃-C₁₄-membered heterocyclic group optionally substituted by R¹⁴,
or R³ and R⁴ with the nitrogen atom to which they are attached form a C₁-C₁₄-membered heterocyclic group optionally substituted by R¹⁴;
L is selected from: 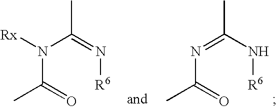
R⁶, R⁵ and R^{x} are selected from H and C₁-C₈ alkyl, C₁-C₈-alkyl-carboxy, C₁-C₈-alkyl-alkoxy, C₁-C₈-haloalkyl, C₃-C₁₅-carbocyclic group, C₁-C₈-alkylcarbonyl, C₁-C₈-alkoxycarbonyl, nitro, cyano, a C₆-C₁₅-membered aromatic carbocyclic group, a 3- to 14-membered heterocyclic group, a C₁-C₈-alkyl substituted by a 3- to 14-membered heterocyclic group, and a C₁-C₈-alkyl substituted by a C₆-C₁₅-membered aromatic carbocyclic group,
W is selected from C₁-C₇ alkylene,
X is selected from —NR⁷(C═O)—,
—NR⁷(C═O)NR⁷—,
—NR⁸SO₂—,
—NR⁸(SO₂)NR⁸—,
—NR⁷(C═O)O—,
—O(C═O)—,
—O(C═O)O—,
—O(C═O)NR⁷—,
—(C═O)NR⁷—,
—(C═O)O—,
—(SO₂)NR⁸—, and
—(SO₂)NR⁸-Z-(SO₂)NR⁸;
Y is —C₀-C₈ alkylene- or (C₀-C₈-alkylene)-SO₂NH—;
Z is C₁-C₄ alkylene;
where W, Y and Z are optionally substituted by C₁-C₈-alkyl, halogen, C₁-C₈-alkoxy, carboxy, C₁-C₈-alkyl-carboxy, C₁-C₈-haloalkyl, C₁-C₈-haloalkoxy, C₃-C₁₅-carbocylic group, C₁-C₈-alkylcarbonyl, C₁-C₈-alkoxycarbonyl, nitro, cyano, a C₃-C₁₅-carbocyclic group, a C₆-C₁₅-membered aromatic carbocyclic group, a C₁-C₈-alkyl substituted by a C₆-C₁₅-membered aromatic carbocyclic group, a 3- to 14-membered heterocylic group containing at least one ring heteroatom selected from the group consisting of nitrogen, oxygen and sulphur, and a C₁-C₈-alkyl substituted by a 4 to 14-membered heterocyclic group containing at least one ring heteroatom selected from the group consisting of nitrogen, oxygen and sulphur; 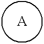
is a C₆-C₁₅-membered aromatic carbocyclic group and a 4- to 14-membered heterocyclic group;
R⁷, R⁸, R¹¹ and R¹², are independently selected from H, C₁-C₈-alkyl, C₁-C₈-alkyl substituted by a C₆-C₁₅-membered aromatic carbocylic group, C₁-C₈-haloalkyl and a 5- to 14-membered heterocyclic group; R⁷ and R⁸, independently, by way of a C₁ to C₄ alkyl group can form a bond with a carbon atom of group W or Y to create a 5- to 14-membered heterocyclic group;
T is selected from H, halogen, C₁-C₈ alkyl, C₁-C₈-haloalkyl, C₁-C₈-haloalkoxy, C₃-C₁₅-carbocyclic group, nitro, cyano, a C₆-C₁₅-membered aromatic carbocyclic group, a and a C₁-C₈-alkyl substituted by a C₆-C₁₅-membered aromatic carbocylic group;
wherein each C₆-C₁₅-membered aromatic carbocyclic group and each 4 to 14 membered heterocyclic group, unless otherwise specified is independently optionally substituted by one or more groups selected from OH, C₁-C₈-alkoxy, C₁-C₈-alkyl, halogen, SO₂NR¹¹R¹², hydroxyC₁-C₈-alkoxy, optionally substituted by hydroxyl, (C₀₋₄alkylene) CONR¹¹R¹², (C₀₋₄alkylene) N═C(NR¹¹R¹²)₂, —O—(C₁₋₄alkylene)-N═C(NR¹¹R¹²)₂, —O—(C₁₋₄alkylene)-CONR¹¹R¹², C₆-C₁₁-aralkoxy, C₁-C₁₀-aralkyl, SH, S(C₁₋₈alkylene), SO₂ (C₁₋₈alkylene) SO(C₁₋₈alkylene), NR¹¹R¹², R¹⁵, a C₁-C₈-alkyl substituted by R¹⁵, R¹⁶, a C₁-C₈-alkyl substituted by R¹⁶, O(C₁-C₈-alkylenyl)-NR¹¹C(C═O)O—(C₀-C₄-alkylene)-R¹⁵, cyano, oxo, carboxy, nitro, C₁-C₈-alkylcarbonyl, hydroxy-C₁-C₈-alkyl, C₁-C₈-haloalkyl, amino-C₁-C₈-alkyl, amino(hydroxy)C₁-C₈-alkyl and C₁-C₈-alkoxy optionally substituted by aminocarbonyl;
and wherein each alkylene group, unless otherwise specified, is optionally substituted by C₁-C₈-alkyl, halogen, C₁-C₈-alkoxy, carboxy, C₁-C₈-alkylcarboxy, C₁-C₈-haloalkyl, C₁-C₈-haloalkoxy, C₃-C₁₅-carbocyclic group, C₁-C₈-alkylcarbonyl, C₁-C₈-alkoxycarbonyl, nitro, cyano, R¹⁵, a C₁-C₈-alkyl substituted by R¹⁵, R¹⁶ or a C₁-C₈-alkyl substituted by R¹⁶;
R¹⁴ is selected from H, halogen, C₁-C₈-alkyl, OH, C₆-C₁₅-membered aromatic carbocyclic group, C₇-C₁₄-aralkyl, and O—C₇-C₁₄-aralkyl;
R¹⁵ is a C₆-C₁₅-membered aromatic carbocyclic group, optionally substituted by OH, C₁-C₈-alkoxy, C₁-C₈-alkyl, halogen and C₁-C₈-haloalkyl; and
R¹⁶ is a 3 to 14 membered heterocyclic group, optionally substituted by OH, C₁-C₈-alkoxy, C₁-C₈-alkyl, halogen and C₁-C₈-haloalkyl.

2. A compound of formula (I) according to claim 1, or tautomers, or stereoisomers,
or pharmaceutically acceptable salts thereof,
wherein
R¹, R², R³, and R⁴ are independently selected from H, C₁-C₈-alkyl, C₁-C₈-alkyl-carboxy;
L is selected from: 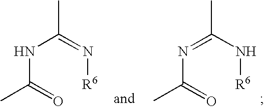
R⁵ and R⁶ are selected from H and C₁-C₈ alkyl;
W is selected from C₁-C₇ alkylene;
X is selected from —NR⁷(C═O)—,
—NR⁷(C═O)NR⁷—,
—NR⁸SO₂—,
—NR⁸(SO₂)NR⁸—,
—NR⁷(C═O)O—,
—O(C═O)—,
—O(C═O)O—,
—O(C═O)NR⁷—,
—(C═O)NR⁷—,
— (C═O)O—,
—(SO₂)NR¹⁸—, and
—(SO₂)NR⁸-Z-(SO₂)NR⁸—
Y is selected from —C₀-C₈ alkylene- or C₀-C₈-alkylene)-SO₂NH—;
Z is C₁-C₄ alkylene; 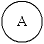
is selected from a C₆-C₁₅-membered aromatic carbocyclic group and a 3 to 14-membered heterocyclic group;
R⁷, R⁸, R¹¹ and R¹², are independently selected from H, C₁-C₈-alkyl, C₁-C₈-haloalkyl, a 5- to 14-membered heterocyclic group, and R⁷ and R⁸, independently, by way of an C₁ to C₄ alkyl group can form a bond with a carbon atom of group W or Y creating a 5- to 14-membered heterocyclic group;
T is selected from H, halogen, C₁-C₈ alkyl, C₁-C₈-haloalkyl, C₁-C₈-haloalkoxy, C₃-C₁₅-carbocyclic group, nitro, cyano, a C₆-C₁₅-membered aromatic carbocyclic group, and a C₁-C₈-alkyl substituted by a C₆-C₁₅-membered aromatic carbocyclic group;
wherein each C₆-C₁₅-membered aromatic carbocyclic group and each 4 to 14 membered heterocyclic group, unless otherwise specified is independently optionally substituted by one or more groups selected from OH, C₁-C₈-alkoxy, C₁-C₈-alkyl, halogen, SO₂NR¹¹R¹², hydroxyC₁-C₈-alkoxy, optionally substituted by hydroxyl, (C₀₋₄alkylene) CONR¹¹R¹², (C₀₋₄alkylene) N═C(NR¹¹R¹²)₂, —O—(C₁₋₄alkylene)-N═C(NR¹¹R¹²)₂, —O—(C₁₋₄alkylene)-CONR¹¹R¹², C₆-C₁₀-aralkoxy, C₇-C₁₀-aralkyl, SH, S(C₁₋₈alkylene), SO₂ (C₁₋₈alkylene) SO(C₁₋₈-alkylene), NR¹¹R¹², R¹⁵, a C₁-C₈-alkyl substituted by R¹⁵, R¹⁶, a C₁-C₈-alkyl substituted by R¹⁶, O(C₁-C₈-alkylene)-NR¹¹C(C═O)O—(C₀-C₄-alkylene)-R¹⁵, cyano, oxo, carboxy, nitro, C₁-C₈-alkylcarbonyl, hydroxy-C₁-C₈-alkyl, C₁-C₈-haloalkyl, amino-C₁-C₈-alkyl, amino(hydroxy)C₁-C₈-alkyl and C₁-C₈-alkoxy optionally substituted by aminocarbonyl;
and wherein each alkylene group, unless otherwise specified, is optionally substituted by C₁-C₈-alkyl, halogen, C₁-C₈-alkoxy, carboxy, C₁-C₈-alkyl-carboxy, C₁-C₈-haloalkyl, C₁-C₈-haloalkoxy, C₃-C₁₅-carbocyclic group, C₁-C₈-alkylcarbonyl, C₁-C₈-alkoxycarbonyl, nitro, cyano, R¹⁵, a C₁-C₈-alkyl substituted by R¹⁵, R¹⁶ or a C₁-C₈-alkyl substituted by R¹⁶;
R¹⁵ is a C₆-C₁₅-membered aromatic carbocyclic group, optionally substituted by OH, C₁-C₈-alkoxy, C₁-C₈-alkyl, halogen and C₁-C₈-haloalkyl; and
R¹⁶ is a 3 to 14 membered heterocyclic group, optionally substituted by OH, C₁-C₈-alkoxy, C₁-C₈-alkyl, halogen and C₁-C₈-haloalkyl.

3. A compound of formula (I) according to claim 1, or tautomers, or stereoisomers, or pharmaceutically acceptable salts thereof,
wherein
R¹, R², R³, R⁴ and R⁵ are H;
L is selected from: 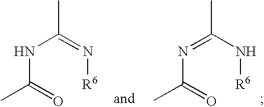
R⁶ is H;
W is selected from C₁-C₇ alkylene;
X is selected from —NR⁷(C═O)—,
—NR⁷(C═O)NR⁷—,
—NR⁸SO₂—,
—NR⁸(SO₂)NR⁸—,
—NR⁷(C═O)O—,
—O(C═O)—,
—O(C═O)O—,
—O(C═O)NR⁷—,
—(C═O)NR⁷—,
—(C═O)O—,
—(SO₂)NR¹⁸—, and
—(SO₂)NR⁸-Z-(SO₂)NR⁸—;
Y is selected from —C₀-C₈ alkylene or —C₀-C₈-alkylene)-SO₂NH—;
Z is C₁-C₄ alkylene; 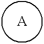
is selected from a C₆-C₁₅-membered aromatic carbocyclic group and a 3- to 14-membered heterocyclic group;
R⁷ and R⁸ are H, or R⁷ and R⁸, independently, by way of an C₁ to C₄ alkyl group can form a bond with a carbon atom of group W or Y creating a 5 to 14-membered heterocyclic group;
R¹¹ and R¹² are independently selected from C₁-C₈-alkyl, C₁-C₈-haloalkyl and a 5 to 14-membered heterocyclic group;
T is a halogen;
wherein each C₆-C₁₅-membered aromatic carbocyclic group and each 3 to 14 membered heterocyclic group, unless otherwise specified is independently optionally substituted by one or more groups selected from OH, C₁-C₈-alkoxy, C₁-C₈-alkyl, halogen, SO₂NR¹¹R¹², hydroxyC₁-C₈-alkoxy, optionally substituted by hydroxyl, (C₀₋₄alkylene) CONR¹¹R¹², (C₀₋₄alkylene) N═C(NR¹¹R¹²)₂, —O—(C₁₋₄alkylene)-N═C(NR¹¹R¹²)₂, —O—(C₁₋₄alkylene)-CONR¹¹R¹², C₆-C₁₀-aralkoxy, C₁-C₁₀-aralkyl, NR¹¹R¹², R¹⁵, a C₁-C₈-alkyl substituted by R¹⁵, R¹⁶, a C₁-C₈-alkyl substituted by R¹⁶, O(C₁-C₈-alkylene)-NR¹¹C(C═O)O—(C₀-C₄-alkylene)-R¹⁵, cyano, oxo, carboxy, nitro,
C₁-C₈-alkylcarbonyl, hydroxy-C₁-C₈-alkyl, C₁-C₈-haloalkyl, amino-C₁-C₈-alkyl, amino(hydroxy)C₁-C₈-alkyl and C₁-C₈-alkoxy optionally substituted by aminocarbonyl;
and wherein each alkylene group, unless otherwise specified, is optionally substituted by C₁-C₈-alkyl, halogen, C₁-C₈-alkoxy, carboxy, C₁-C₈-alkyl-carboxy, C₁-C₈-haloalkyl, C₁-C₈-haloalkoxy, C₃-C₁₅-carbocyclic group, C₁-C₈-alkylcarbonyl, C₁-C₈-alkoxycarbonyl, nitro, cyano, R¹⁵, a C₁-C₈-alkyl substituted by R¹⁵, R¹⁶ or a C₁-C₈-alkyl substituted by R¹⁶;
R¹⁵ is a C₆-C₁₅-membered aromatic carbocyclic group, optionally substituted by OH, C₁-C₈-alkoxy, C₁-C₈-alkyl, halogen and C₁-C₈-haloalkyl; and
R¹⁶ is a 3 to 14 membered heterocyclic group, optionally substituted by OH, C₁-C₈-alkoxy, C₁-C₈-alkyl, halogen and C₁-C₈-haloalkyl.

4. A compound according to claim 1, wherein said compound is selected from:

| Ex. | Structure |
|---|---|
| 1 | 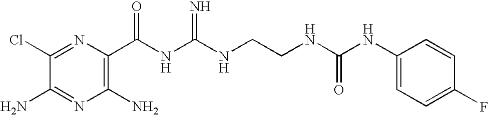 |
| 2 | 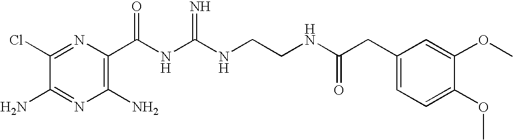 |
| 3 | 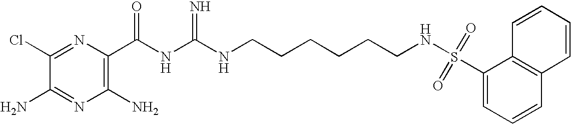 |
| 4 | 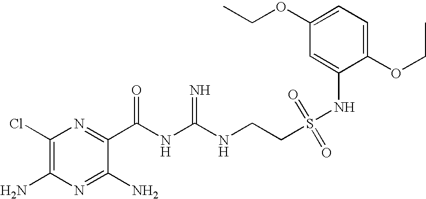 |
| 5 | 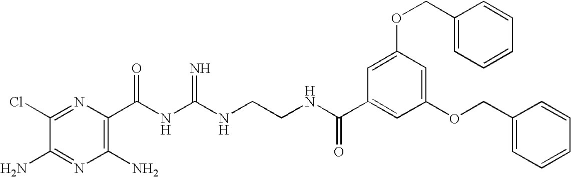 |
| 6 | 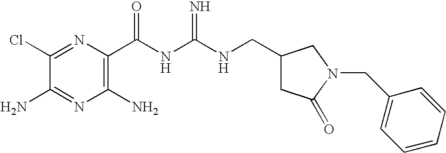 |
| 7 | 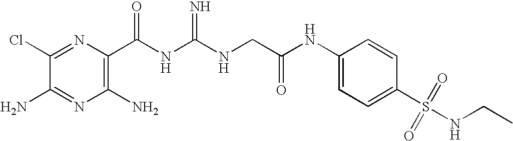 |
| 8 | 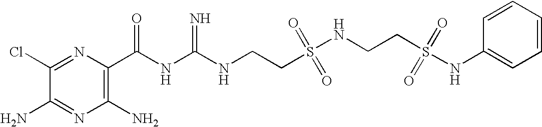 |
| 9 | 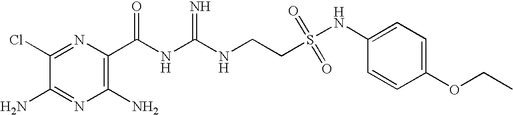 |
| 10 | 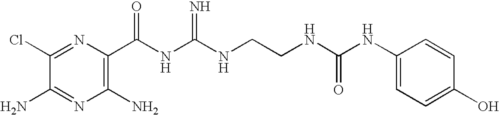 |
| 11 | 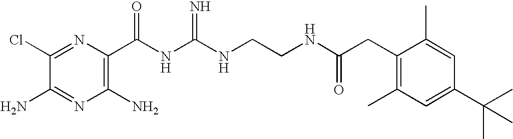 |
| 12 | 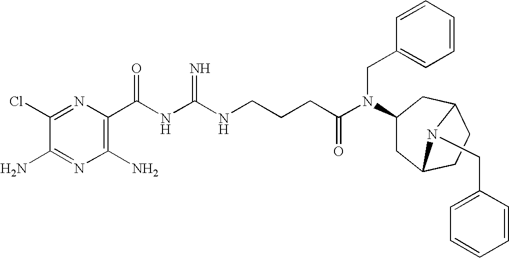 |
| 13 | 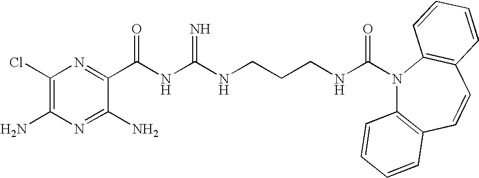 |
| 14 | 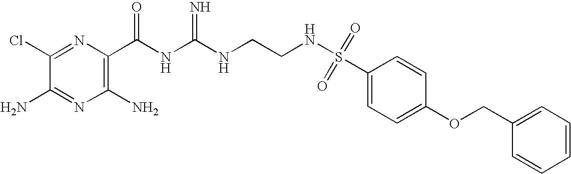 |
| 15 | 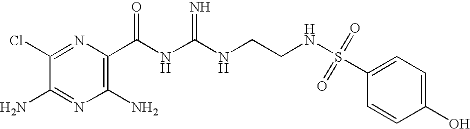 |
| 16 | 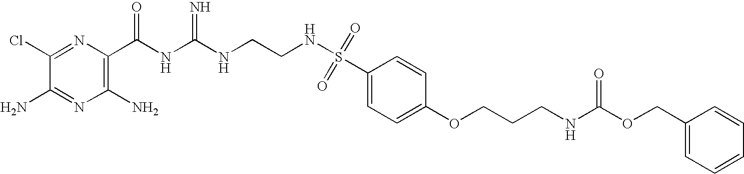 |
| 17 | 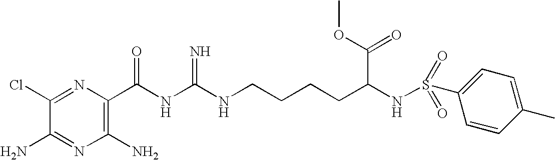 |
| 18 | 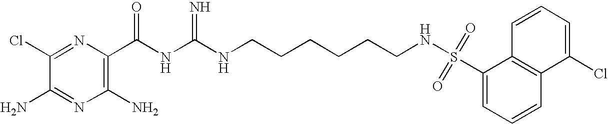 |
| 19 | 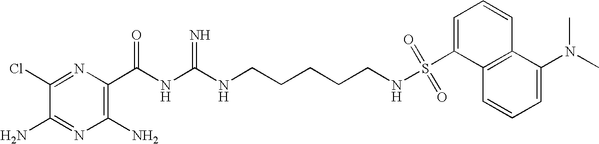 |
| 20 | 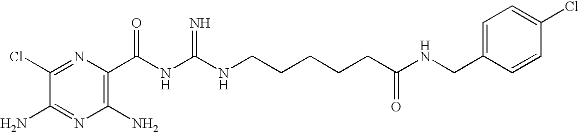 |
| 21 | 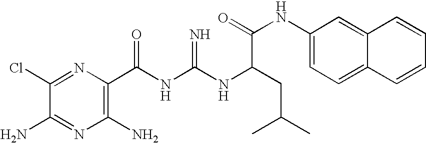 |
| 22 | 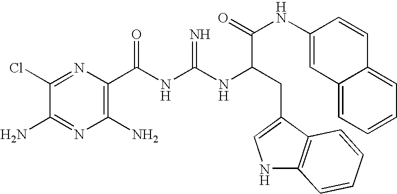 |
| 23 | 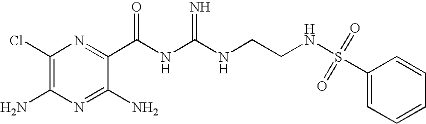 |
| 24 | 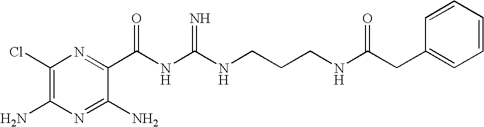 |
| 25 | 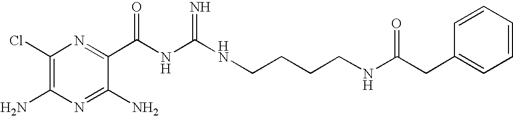 |
| 26 | 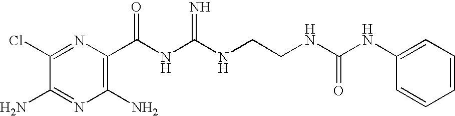 |
| 27 | 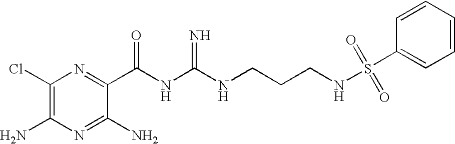 |
| 28 | 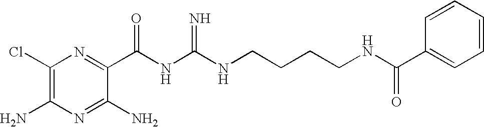 |
| 29 | 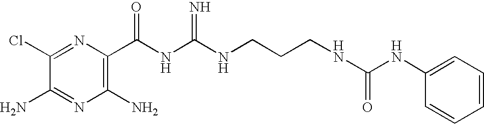 |
| 30 | 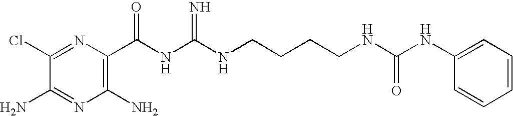 |
| 31 | 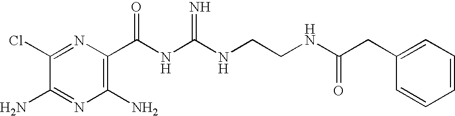 |
| 32 | 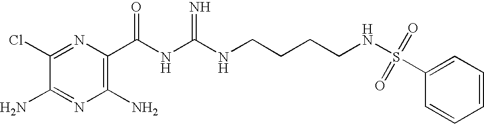 |
| 33 | 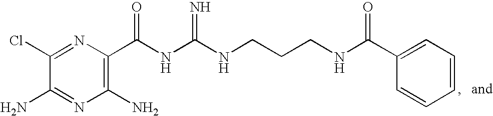 |
| 34 | 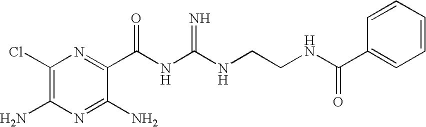 |

5. A compound according to claim 1 for use as a pharmaceutical.

6. Pharmaceutical compositions comprising a compound according to claim 1.

7. The use of a compound according to claim 1, in the manufacture of a medicament for treatment of a disease mediated by the blockade of an epithelial sodium channel.

8. The use of a compound according to claim 1, in the manufacture of a medicament for treatment of an inflammatory or allergic condition, particularly an inflammatory or obstructive airways disease.

9. The use of a compound according to claim 1, in the manufacture of a medicament for the treatment of an inflammatory or allergic condition selected from cystic fibrosis, primary ciliary dyskinesia, chronic bronchitis, chronic obstructive pulmonary disease, asthma, respiratory tract infections, lung carcinoma, xerostomia, and keratoconjunctvitis sire.

10. A combination of a compound according to claim 1 with an anti-inflammatory, bronchodilatory, antihistamine or anti-tussive drug substance.

11. A process for the preparation of compounds of formula (I) 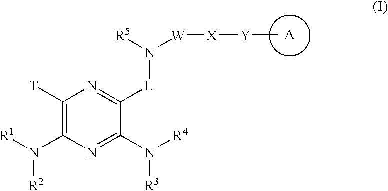
wherein R¹, R², R³, R⁴, R⁵, T, L, W, X, Y, and 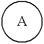
are as defined hereinbefore, which comprises the steps of:
(i) reacting a compound of formula (IV) 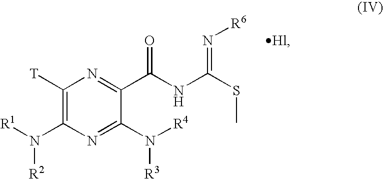
wherein R¹, R², R³, R⁴, R⁶ and T are as hereinbefore defined,
with compounds of formula (V) 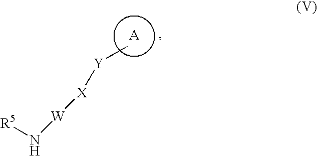
wherein R⁵, W, X, Y, and 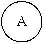
are hereinbefore defined,
optionally in the presence of a base, e.g., an organic base; and in an organic solvent, e.g., a non-protic dipolar solvent; and
(ii) recovering the resultant compound of formula (I), in free or pharmaceutically acceptable salt form.
